(19) United States
(12) Patent Application Publication
(10) Pub. No.: US 2010/0092534 A1
(43) Pub. Date: Apr. 15, 2010

(54) Combination Local Delivery Using a Stent

(75) Inventors: Ayala Hezi-Yamit, Windsor, CA (US); Iskender Matt Bilge, San Francisco, CA (US); Jennifer Wong, Santa Rosa, CA (US); Carol Sullivan, Fairfax, CA (US); Natividad Vasquez, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/249,576

(22) Filed: Oct. 10, 2008

(51) Int. Cl. A61F 2/04; A61K 31/58; A61K 31/4355

(52) U.S. Cl. 424/423; 514/171; 514/391

(57) ABSTRACT

Described herein are implantable medical devices useful in treating vascular conditions such as restenosis. In one embodiment, stents are described in which a combination of bioactive agents is described for local delivery in the vasculature. The combination of bioactive agents comprises at least one compound capable of inhibiting smooth muscle cell proliferation and at least one compound capable of mitigating MCP- and/or TF induction. For example, a compound capable of inhibiting smooth muscle cell proliferation is a mTOR inhibitor and a compound capable of mitigating MCP-1 and/or TF induction is a corticosteroid.

### FIELD OF THE INVENTION

The present invention relates to the administration of novel bioactive agent combinations in conjunction with vascular stent therapy.

### BACKGROUND OF THE INVENTION

Deployment of vascular stents into occluded vasculature is a remedy for stenosis. Since its inception into the medical field, the stent has been refined in a number of ways, from building materials, to polymer coatings, to bioactive agent/drug integration. For over a decade, stents, in their various forms, have prolonged the life of thousands of patients. However, there have been problems associated with using stents to treat vascular conditions. One major problem with vascular stents, in particular, is the occurrence of restenosis.

Restenosis is taken literally to mean re-occurrence of stenosis. An important cause of restenosis is the inflammatory response which induces tissue proliferation around an angioplasty site. The paradigm of restenosis is based, at least in part, on the vascular biology of wound healing.

There are three phases understood in the process of wound healing. The three phases are as follows, an inflammatory phase, a cellular proliferation phase, and a phase of remodeling involving extracellular matrix protein synthesis. Therefore, in order to prevent restenosis, methods need to be developed to reduce inflammation, reduce proliferation of cells, reduce extracellular matrix protein synthesis, or some combination of the three.

### SUMMARY OF THE INVENTION

Described herein are implantable medical devices useful in treating vascular conditions such as restenosis. In one embodiment, stents are described in which a combination of bioactive agents is described for local delivery in the vasculature. The combination of bioactive agents comprises at least one compound capable of inhibiting smooth muscle cell proliferation and at least one compound capable of mitigating monocyte chemoattractant protein-1 (MCP-1) and/or tissue factor (TF) induction. In one embodiment, the compound capable of inhibiting smooth muscle cell proliferation is an mTOR inhibitor and the compound capable of mitigating MCP-1 and/or TF induction is a glucocorticoid.

In one embodiment described herein a stent is described comprising (a) a predominantly cylindrical shape comprising an inner surface, an outer surface, a proximal end and a distal end; (b) at least one polymer covering at least a portion of the inner surface, the outer surface, the proximal end, or said distal end; (c) at least one compound capable of inhibiting smooth muscle cell proliferation dispersed within the polymer; and (d) at least one compound capable of mitigating MCP-1 and/or TF induction dispensed within the polymer coating. In another embodiment, the compound capable of mitigating MCP-1 and/or TF induction is a corticosteroid such as a glucocorticoid. In one embodiment, the corticosteroid is fluocinolone.

In one embodiment, the compound capable of inhibiting smooth muscle cell proliferation is a mTOR inhibitor. In one embodiment, the mTOR inhibitor is selected from the group consisting of sirolimus, everolimus, and zotarolimus.

In one embodiment, the stent is selected from the group consisting of vascular stents, urethral stents, biliary stents, or stents intended for use in other ducts and organ lumens. In another embodiment, the stent has a core structure comprising a metal, a metal alloy, a polymer, a polymer blend, a polymer matrix, or combinations thereof.

In one embodiment, the polymer is selected from the group consisting of polyolefins, polyisobutylene, ethylene-alphaolefin copolymers, acrylic polymers, acrylic copolymers, ethylene-co-vinylacetate, polybutylmethacrylate, vinyl halide polymers, vinyl halide copolymers, polyvinyl ethers, polyvinylidene halides, polyacrylonitrile, polyvinyl ketones, polyvinyl aromatics, polyvinyl esters, polyvinyl amides such as polyvinyl pyrrolidone, copolymers of vinyl monomers with each other, copolymers of vinyl monomers with olefins, acrylonitrile-styrene copolymers, polyamides, alkyd resins, polycarbonates, polyoxymethylenes, polyimides, polyethers; epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, and combinations thereof.

In one embodiment, the at least one compound capable of inhibiting smooth muscle cell proliferation is present at about 0 to 1000 μg. In one embodiment, the at least one compound capable of mitigating MCP-1 and/or TF induction is present at about 0 to 1000 μg. In one embodiment, the polymer and the at least one compound capable of inhibiting smooth muscle cell proliferation have a ratio of about 5:1. In another embodiment, the polymer and the at least one compound capable of mitigating MCP-1 and/or TF induction have a ratio of about 5:1.

In one embodiment, a method is described of forming a bioactive stent comprising the steps of (a) providing a stent; (b) providing at least one polymer; (c) providing at least one compound capable of inhibiting smooth muscle cell proliferation and at least one compound capable of mitigating MCP-1 and/or TF induction; (d) combining the at least one compound capable of inhibiting smooth muscle cell proliferation and at least one compound capable of mitigating MCP-1 and/or TF induction with the at least one polymer to create a bioactive polymer system; and (e) coating at least a portion of the stent with the bioactive polymer system to form a bioactive stent. In one embodiment, the stent is selected from the group consisting of vascular stents, urethral stents, biliary stents, or stents intended for use in other ducts and organ lumens.

In one embodiment, the polymer is selected from the group consisting of polyolefins, polyisobutylene, ethylene-alphaolefin copolymers, acrylic polymers, acrylic copolymers, ethylene-co-vinylacetate, polybutylmethacrylate, vinyl halide polymers, vinyl halide copolymers, polyvinyl ethers, polyvinylidene halides, polyacrylonitrile, polyvinyl ketones, polyvinyl aromatics, polyvinyl esters, polyvinyl amides such as polyvinyl pyrrolidone, copolymers of vinyl monomers with each other, copolymers of vinyl monomers with olefins, acrylonitrile-styrene copolymers, polyamides, alkyd resins, polycarbonates, polyoxymethylenes, polyimides, polyethers; epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, and combinations thereof.

In one embodiment, the at least one compound capable of inhibiting smooth muscle cell proliferation is a mTOR inhibitor. In one embodiment, the mTOR inhibitor is selected from the group consisting of sirolimus, everolimus, and zotarolimus. In another embodiment, the at least one compound capable of mitigating MCP-1 and/or TF induction is a glucocorticoid. In one embodiment, the glucocorticoid is fluocinolone.

In one embodiment, the lumen is a coronary artery. In another embodiment, the at least one compound capable of inhibiting smooth muscle cell proliferation is present at about 0 to 1000 μg. In one embodiment, the at least one compound capable of mitigating MCP-1 and/or TF induction is present at about 0 to 1000 μg. In another embodiment, the polymer and the at least one compound capable of inhibiting smooth muscle cell proliferation have a ratio of about 5:1. In yet another embodiment, the polymer and the at least one compound capable of mitigating MCP-1 and/or TF induction have a ratio of about 5:1.

### BRIEF DESCRIPTION OF THE FIGURES

FIG. 1: Relative gene expression of (A) MCP-1 and (B) TF in porcine vessels that were stented with zotarolimus coated stents compared with uncoated stents and stents coated with fluocinolone. The drug doses are indicated in ug/mm².

FIG. 2: Relative gene expression of MCP-1 in porcine vessels that were stented with stents coated with zotarolimus alone compared to stents coated with zotarolimus combined with fluocinolone or fluocinolone alone. The drug doses are indicated in ug/mm².

FIG. 3: Hypothetical results illustrating experiment designed for identification of optimal dosage for combination of zotarolimus (e.g. anti-proliferative drug) and fluocinolone (e.g. anti-inflammatory drug), (A) via evaluation of MCP-1 expression and (B) via evaluation of TF expression. When indicated, the drug doses are indicated in ug/mm².

### DEFINTIONS

Bioactive Agent: As used herein “bioactive agent” shall include any drug, pharmaceutical compound or molecule having a therapeutic effect in an animal. The use of drug herein falls within the scope of bioactive agent. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes. Bioactive agents can also include steroids, including glucocorticoids and/or corticosteroids.

Exemplary FKBP 12 binding compounds include mTOR inhibitors such as, but not limited to sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, and other rapamycin hydroxyesters may be used in combination with the terpolymers of the present invention.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to a polymeric composition that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

Nonbiodegradable: As used herein “nonbiodegradable” refers to a polymeric composition that is biocompatible and not subject to being broken down in vivo through the action of normal biochemical pathways.

Substantially Non-Toxic: As used herein “substantially non-toxic” shall mean systemic or localized toxicity wherein the benefit to the recipient is out-weighted by the physiologically harmful effects of the treatment as determined by physicians and pharmacologists having ordinary skill in the art of toxicity.

Pharmaceutically Acceptable: As used herein “pharmaceutically acceptable” refers to all derivatives and salts that are not substantially toxic at effective levels in vivo.

### DETAILED DESCRIPTION OF THE INVENTION

The most common mechanism of action currently used to combat restenosis from vascular stent treatment is the inhibition of smooth muscle cell proliferation. This inhibition is attained by systemic delivery of a bioactive agent capable of inhibiting smooth muscle cell proliferation or by local delivery of the bioactive agent from a stent. More specifically, bioactive agents such as inhibitors of mammalian target of rapamycin (mTORs) are known to be potent inhibitors of smooth muscle cell proliferation.

Exemplary mTOR inhibitors may have the following formulae: sirolimus:

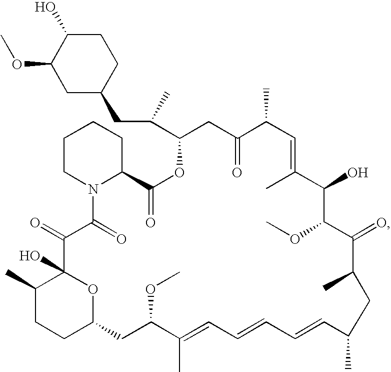

everolimus:

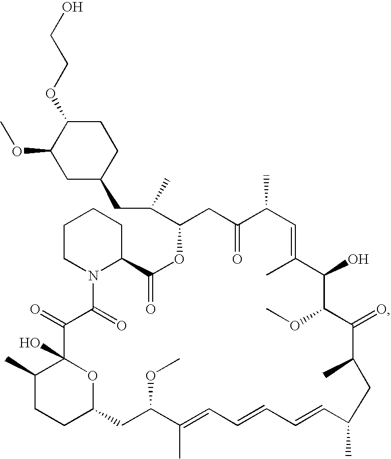

and zotarolimus:

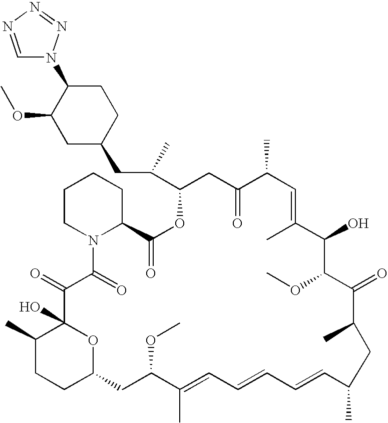

have shown consistent effects in reducing restenosis in clinical as well as pre-clinical (porcine) studies.

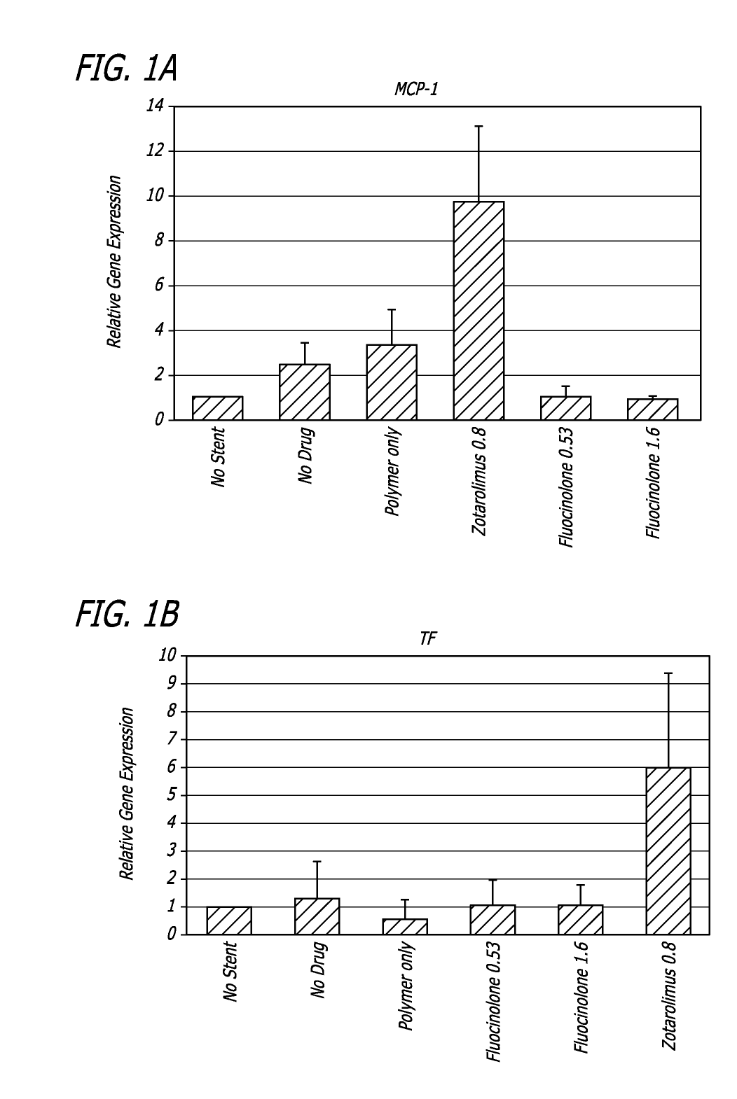

However, in recent pre-clinical studies, the inventors unexpectedly found that delivery of zotarolimus locally from a vascular stent increased the expression of monocyte chemoattractant protein-1 (MCP-1) in stented vessels 7 days after the stenting procedure was performed, as presented in FIG. 1A (the increase in expression was compared to a bare metal stent and was measured on mRNA). Furthermore, following stent placement, MCP-1 levels in plasma increase after several days and are more likely to be elevated at follow-up 6 months later in patients who have restenosis.

MCP-1 belongs to the subfamily of C-C chemokines-beta and is responsible for the direct migration of monocytes into the intima at sites of lesion formation. In addition to promoting the transmigration of circulating monocytes into tissues, MCP-1 exerts various other effects on monocytes, including superoxide anion induction, cytokine production and adhesion molecule expression. Inflammatory cytokines or peptide growth factors induce MCP-1 expression in endothelial cells or vascular smooth muscle cells. Since elevated levels of MCP-1 have been demonstrated in myocardial infarction, heart failure and after angioplasty, this chemokine is probably a key factor in the initiation of the inflammatory process and maintaining the proliferative response to vascular injury restenosis. In addition, MCP-1 can contribute to thrombin generation and thrombus formation by inducing expression and generating tissue factor (TF) in the vessel wall resident cells and by adhering monocytes.

Tissue factor (also commonly referred to as TF, coagulation factor III, CD142) is a 46-kDa transmembrane glycoprotein that serves as one of the primary initiators of blood coagulation. Cell-anchored TF interacts with soluble factor VIIa (FVIIa) to induce factor Xa (FXa) activation, leading to cleavage of prothrombin to thrombin the proteolytically active protease. Thrombin in turn is responsible for conversion of plasma fibrinogen to fibrin, which envelopes and stabilizes developing thrombi (blood clots). Thrombin also cleaves and activates the platelet receptor PAR1 (protease-activated receptor 1; also known as the thrombin receptor), which induces platelet aggregation and thrombus growth.

As such, delivery of zotarolimus locally from a vascular stent increases the expression of TF in stented vessels. The increased expression can be seen several days after the stenting procedure was preformed (FIG. 1B).

In healthy vessels, mTOR inhibition induced MCP-1 elevation after stenting is less likely to lead to increased neointimal hyperplasia than in at-risk vessels. In at-risk vessels, for example in elderly and diabetic patients, MCP-1 and TF induction may be of concern as inflammation is established to underline cardiovascular complications.

In one embodiment, the local delivery of a compound capable of inhibiting smooth muscle cell proliferation herein can be an mTOR inhibitor, in conjunction with at least one compound or bioactive agent that can mitigate the induction of MCP-1 and TF in coronary arteries following stenting is described.

In one embodiment, the bioactive agent capable of mitigation of the induction of MCP-1 and/or TF can include, but is not limited to any molecule that can inhibit the expression, the secretion or the action of MCP-1. These may include small synthetic molecules, small naturally occurring molecules, or neutralizing proteins /antibodies or MCP-1 gene silencing molecules (such as viral DNA, or anti-sense oligonucleotides or inhibitory siRNA).

In one embodiment, the bioactive agent capable of mitigation of the induction of MCP-1 and/or TF can include anti-inflammatory compounds such as steroids. More specifically, the steroids can include glucocorticoids and/or corticosteroids which have anti-inflammatory effects, irrespective of their cause. The two major products of inflammation, prostaglandins and leukotrienes are inhibited by corticosteroids such as glucocorticoids.

In one embodiment, the corticosteroid can include, but is not limited to, deflazacort, budesonide, beclomethasone dipropionate, cortisol, hydrocortisone, rimexolone, loteprednol etabonate (non-ester loteprednol), salmeterol xinafoate, fluticasone propionate, etiprednol dichloroacetate, prednisolone, dexamethasone, triamcinolone acetonide, clocortolone pivalate, and fluocinolone.

In one embodiment, the corticosteroid can be fluocinolone. Fluocinolone has the following structure:

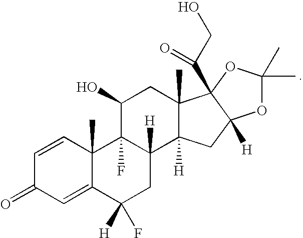

Fluocinolone, sometimes referred to as fluocinolone acetonide, is derivative of hydrocortisone and can be used in dermatology to treat inflammation and itching.

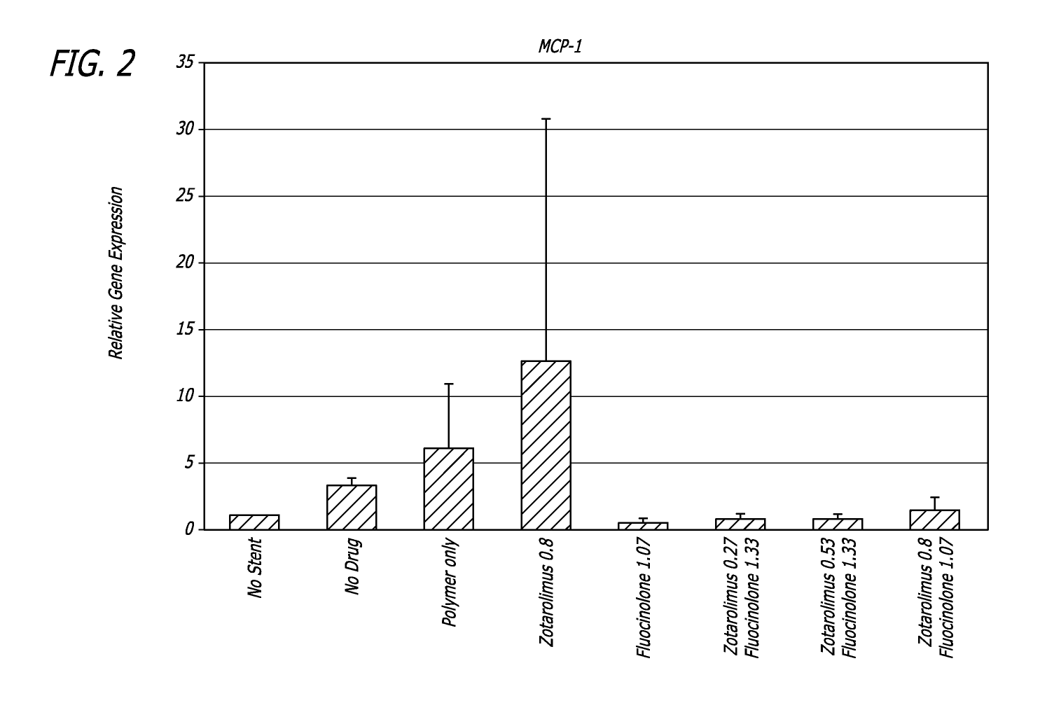

The combination local delivery of mTOR inhibitors with compounds that can mitigate the induction of MCP-1 (FIG. 2) to the coronary vasculature provides an inhibition of smooth muscle cell proliferation while providing reduced induction of MCP-1 and TF. With such a combined treatment, the patient receives the beneficial effects of mTOR inhibitors with the low activity of MCP-1 and TF thereby reducing inflammation at the stenting site.

In one embodiment, the stent has dispersed on it or within a coating on it, at least zotarolimus (an mTOR inhibitor) and fluocinolone (a mitigator of MCP-1 and TF activity). The two bioactive agents can be dispersed within a polymeric coating or within multiple polymeric coatings. If the stent itself is constructed of polymeric material, the bioactive agents can be dispersed within that material, or within any applied polymeric coatings.

It will be understood by those skilled in the art, that the mTOR inhibitors and MCP-1/TF mitigators discussed herein are a few of the many pharmaceutically acceptable bioactive agents that fulfill the requirements necessary to achieve the desired benefits described herein. Many other pharmaceutically acceptable forms can be synthesized and are still considered to be within the scope of the present description. Moreover, many derivatives are also possible that do not affect the efficacy or mechanism of action of the bioactive agents. Therefore, the present description is intended to encompass pharmaceutically acceptable derivatives, salts, prodrugs, and combinations thereof of the bioactive agents described herein.

The bioactive agent combinations discussed herein may be added to implantable medical devices. The bioactive agent combinations may be incorporated into the polymer coating applied to the surface of a medical device or may be incorporated into the polymer used to form the medical device. The bioactive agent combinations may be coated to the surface with or without a polymer using methods including, but not limited to, precipitation, coacervation, and crystallization. The bioactive agent combinations may be bound covalently, ionically, or through other intramolecular interactions, including without limitation, hydrogen bonding and van der Waals forces.

The medical devices used may be permanent medical implants, temporary implants, or removable devices. For example, and not intended as a limitation, the medical devices may include stents, catheters, micro-particles, probes, and vascular grafts.

In one embodiment, the stents may be vascular stents, urethral stents, biliary stents, or stents intended for use in other ducts and organ lumens. Vascular stents, for example, may be used in peripheral, neurological, or coronary applications. The stents may be rigid expandable stents or pliable self-expanding stents. Any biocompatible material may be used to fabricate stents, including, without limitation, metals and polymers. The stents may also be bioresorbable. In one embodiment, vascular stents are implanted into coronary arteries immediately following angioplasty. In another embodiment, vascular stents are implanted into the abdominal aorta to treat an abdominal aneurysm.

In one embodiment, the stent is a vascular stent with a predominantly cylindrical (tubular) shape. The shape can be defined by a longitudinal axis with a proximal end and a distal end. In addition, the stent has an inner surface which can contact the fluids flowing through the vessel of implantation and an outer surface which contacts at least a portion of the surface of the vessel in which the stent is deployed.

In one embodiment, the stent's core is made of metal or metal alloys. In another embodiment, the stent's core is made of a combination of metals and/or metal alloys. The metals and/or metal alloys can be degradable such as magnesium or can be non-erodable such as stainless steel.

In one embodiment, metallic vascular stents are coated with one or more bioactive agent combinations. The bioactive agent combination may be dissolved or suspended in any carrier compound that provides a stable, un-reactive environment for the bioactive agent combination. The stent can be coated with a bioactive agent combination coating according to any technique known to those skilled in the art of medical device manufacturing. Suitable, non-limiting examples include impregnation, spraying, brushing, dipping and rolling. After the bioactive agent combination is applied to the stent, it is dried leaving behind a stable bioactive agent combination delivering medical device. Drying techniques include, but are not limited to, heated forced air, cooled forced air, vacuum drying or static evaporation. Moreover, the medical device, specifically a metallic vascular stent, can be fabricated having grooves or wells in its surface that serve as receptacles or reservoirs for the bioactive agent combinations described herein. In the case of a polymeric stent, grooves and wells can be manufactured into the surface of the device. In addition, pores can be manufactured into the surface of the device, which is a method commonly known to those skilled in the art.

The effective amount of each bioactive agent in the bioactive agent combination as used herein can be determined by a titration process. Titration is accomplished by preparing a series of stent sets. Each stent set will be coated, or contain different dosages of bioactive agent combination. The highest concentration used will be partially based on the known toxicology of the compounds in the bioactive agent combination. The maximum amount of bioactive agent delivered by the stents will fall below known toxic levels. The dosage selected for further studies will be the minimum dose required to achieve the desired clinical outcome. In one embodiment, the desired clinical outcome is defined as a site specific decrease in smooth muscle cell proliferation while mitigating the induction of MCP-1 and/or TF.

In another embodiment, the bioactive agent combination is precipitated or crystallized on or within the stent. In yet another embodiment, the bioactive agent combination is mixed with a suitable biocompatible polymer (bioerodable, bioresorbable, or non-erodable). The polymer-bioactive agent combination blend can then be used to produce a medical device such as, but not limited to, stents, grafts, micro-particles, sutures and probes. Furthermore, the polymer-bioactive agent combination blend can be used to form controlled-release coatings for medical device surfaces. For example, and not intended as a limitation, the medical device can be immersed in the polymer-bioactive agent combination blend, the polymer-bioactive agent combination blend can be sprayed, or the polymer-bioactive agent combination blend can be brushed onto the medical device. In another embodiment, the polymer-bioactive agent combination blend can be used to fabricate fibers or strands that are embedded into the medical device or used to wrap the medical device.

In one embodiment, the polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that can be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters may be used. Other polymers that can be dissolved and cured or polymerized on the medical device may be used, for example, such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; polyvinyl amides such as polyvinyl pyrrolidone, copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer coatings or medical devices formed from polymeric material discussed herein may be designed with a specific dose of bioactive agent combination. That dose may be a specific weight of each bioactive agent added or a ratio of each bioactive agent to polymer. In one embodiment, the medical device can be loaded with 0 to 1000 μg of an mTOR inhibitor and 0 to 1000 μg of a glucocorticoid (glucocorticoid used as a non-limiting example of a mitigator of MCP-1 and/or TF induction); in another embodiment, 0 to 1000 μg of an mTOR inhibitor and 5 to 500 μg of a glucocorticoid; in another embodiment 0 to 1000 μg of an mTOR inhibitor and 10 to 250 of a glucocorticoid; in another embodiment, 0 to 1000 μg of an mTOR inhibitor and 15 to 150 μg of a glucocorticoid; in another embodiment, 5 to 500 μg of an mTOR inhibitor and 0 1000 μg of a glucocorticoid; in another embodiment 10 to 250 μg of an mTOR inhibitor and 0 to 1000 μg of a glucocorticoid; in another embodiment, 15 to 150 μg of an mTOR inhibitor and 0 to 1000 μg of a glucocorticoid. In addition, combinations of the amounts mentioned above of each bioactive agent can be considered within the scope of the present description.

A ratio may also be established to describe how much bioactive agent combination is added to the polymer that is coated to or formed into the medical device. In one embodiment a ratio of 1 part bioactive agent combination: 1 part polymer may be used; in another embodiment, 1:1-5; in another embodiment, 1:1-9; in another embodiment, 1:1-20. In addition, if different amounts of each bioactive agent are used, the ratio can be split into three, for example, 1 part mTOR inhibitor: 0.5 part glucocorticoid: 1 part polymer. One skilled in the art will appreciate that there are countless combinations that can be contemplated and are all considered to be within the scope of the present description.

In addition to the site specific delivery of bioactive agent combinations, the implantable medical devices discussed herein can accommodate one or more additional bioactive agents. The choice of bioactive agent to incorporate, or how much to incorporate, will have a great deal to do with the polymer selected to coat or form the implantable medical device. A person skilled in the art will appreciate that hydrophobic agents prefer hydrophobic polymers and hydrophilic agents prefer hydrophilic polymers. Therefore, coatings and medical devices can be designed for agent or agent combinations with immediate release, sustained release or a combination of the two.

Exemplary, non limiting examples of bioactive agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Bioactive agents can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers described herein.

### EXAMPLES

Providing a Metallic Surface with an mTOR Inhibitor/Glucocorticoid-Eluting Coating

The following Examples are intended to illustrate a non-limiting process for coating metallic stents with an mTOR Inhibitor and glucocorticoid. One non-limiting example of a suitable metallic stent is the Medtronic/AVE S670 ™ 316L stainless steel coronary stent.

### Example 1

### Metal Stent Cleaning Procedure

Stainless steel stents are placed in a glass beaker and covered with reagent grade or better hexane. The beaker containing the hexane immersed stents is then placed into an ultrasonic water bath and treated for 15 minutes at a frequency of between approximately 25 to 50 KHz. Next the stents are removed from the hexane and the hexane is discarded. The stents are then immersed in reagent grade or better 2-propanol and vessel containing the stents and the 2-propanol is treated in an ultrasonic water bath as before. Following cleaning the stents with organic solvents, they are thoroughly washed with distilled water and thereafter immersed in 1.0 N sodium hydroxide solution and treated at in an ultrasonic water bath as before. Finally, the stents are removed from the sodium hydroxide, thoroughly rinsed in distilled water and then dried in a vacuum oven over night at 40° C. After cooling the dried stents to room temperature in a desiccated environment they are weighed their weights are recorded.

### Example 2

### Coating a Clean, Dried Stent Using a Bioactive Agent/Polymer System

In the following Example, ethanol is chosen as the solvent of choice. The coating to be applied to the stent is a bioactive agent combination, consisting of a mTOR inhibitor and a glucocorticoid. The mTOR inhibitor is zotarolimus. The glucocorticoid is fluocinolone. The polymer, zotarolimus and fluocinolone are freely soluble in ethanol. Persons having ordinary skill in the art of polymer chemistry can easily pair the appropriate solvent system to the polymer-bioactive agent(s) combination and achieve optimum results with no more than routine experimentation.

125 mg of zotarolimus and 125mg of fluocinolone are carefully weighed and added to a small neck glass bottle containing 2.8 ml of ethanol. The bioactive agents-ethanol suspension is then thoroughly mixed until a clear solution is achieved.

Next 250 mg of polycaprolactone (PCL) is added to the bioactive agents-ethanol solution and mixed until the PCL dissolved forming a bioactive agents/polymer solution.

The cleaned, dried stents are coated using either spraying techniques or dipped into the bioactive agent/polymer solution. The stents are coated as necessary to achieve a final coating weight of between approximately 10 μg to 1 mg. Finally, the coated stents are dried in a vacuum oven at 50° C. over night. The dried, coated stents are weighed and the weights recorded.

The concentration of bioactive agents loaded onto (into) the stents is determined based on the final coating weight. Final coating weight is calculated by subtracting the stent's pre-coating weight from the weight of the dried, coated stent.

### Example 3

### Coating a Clean, Dried Stent Using a Sandwich-Type Coating

A cleaned, dry stent is first coated with polyvinyl pyrrolidone (PVP) or another suitable polymer followed by a coating of zotarolimus and fluocinolone. Finally, a second coating of PVP is provided to seal the stent thus creating a PVP-bioactive agents-PVP sandwich coated stent.

### The Sandwich Coating Procedure:

100 mg of PVP is added to a 50 mL Erlenmeyer containing 12.5 ml of ethanol. The flask is carefully mixed until all of the PVP is dissolved. In a separate clean, dry Erlenmeyer flask 125 mg of zotarolimus and 125 mg of fluocinolone are added to 11 mL of ethanol and mixed until dissolved.

A clean, dried stent is then sprayed with PVP until a smooth confluent polymer layer was achieved. The stent is then dried in a vacuum oven at 50° C. for 30 minutes.

Next, successive layers of zotarolimus and fluocinolone are applied to the polymer-coated stent. The stent is allowed to dry between each of the successive coats. After the final bioactive agent coating has dried, three successive coats of PVP are applied to the stent followed by drying the coated stent in a vacuum oven at 50° C. over night. The dried, coated stent is weighed and its weight recorded.

The concentration of bioactive agent in the bioactive agent/polymer solution and the final amount of bioactive agent loaded onto the stent determine the final coating weight. Final coating weight is calculated by subtracting the stent's pre-coating weight from the weight of the dried, coated stent.

### Example 4

### Coating a Cleans Dried Stent with Pure Bioactive Agent

0.5 g of zotarolimus and 0.5 g of fluocinolone are carefully weighed and added to a small neck glass bottle containing 12 mL of ethanol. The bioactive agent-ethanol suspension is then heated at 50° C. for 15 minutes and then mixed until the zotarolimus and fluocinolone are completely dissolved.

Next a clean, dried stent is mounted over the balloon portion of angioplasty balloon catheter assembly. The stent is then sprayed with, or in an alternative embodiment, dipped into, the bioactive agent-ethanol solution. The coated stent is dried in a vacuum oven at 50° C. over night. The dried, coated stent was weighed and its weight recorded.

The concentration of bioactive agent loaded onto (into) the stents is determined based on the final coating weight. Final coating weight is calculated by subtracting the stent's pre-coating weight from the weight of the dried, coated stent.

### Example 5

### Abdominal Aneurysm

In one embodiment, a stent loaded with at least zotarolimus and fluocinolone can be used to deliver the bioactive agents locally to the abdominal aorta.

### Example 6

### Local Delivery to Coronary Artery

In one embodiment, a stent loaded with at least zotarolimus and fluocinolone can be used to deliver the bioactive agents locally to the coronary artery.

### Example 7

### Dose Titration Porcine Studies

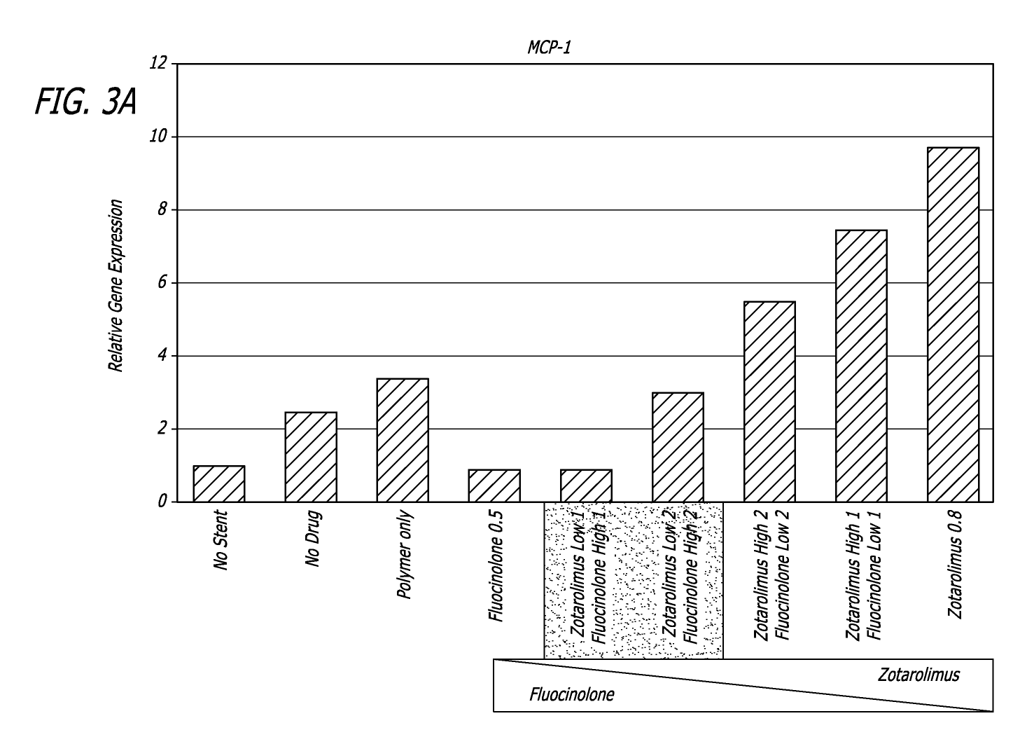

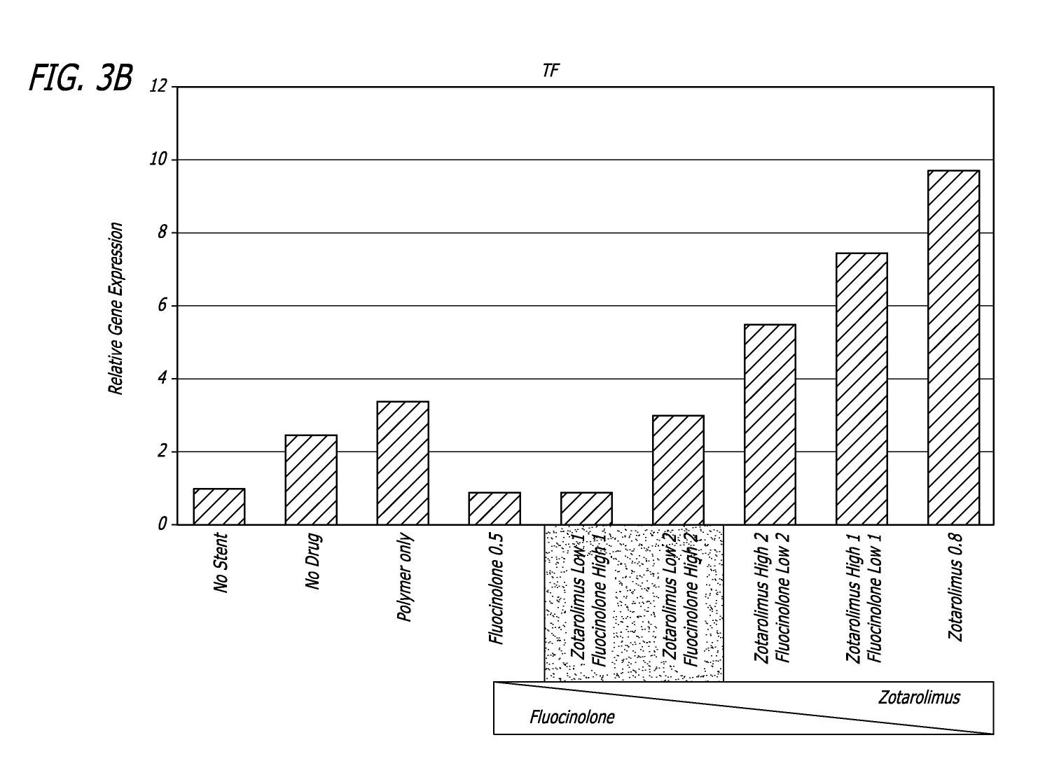

In one embodiment, a dose titration experiment can be conducted to determine the optimal dosage for combination of zotarolimus (e.g. anti-proliferative drug) and fluocinolone (e.g. anti-inflammatory drug). At an optimal dose zotarolimus can inhibit neointimal hyperplasia via inhibition of smooth muscle cells proliferation, while maintaining low levels of MCP-1 and TF due to the inhibitory effect of fluocinolone. The evaluation of MCP-1 and TF can be performed by conducting pre-clinical, porcine study using stents loaded with escalating doses of at least zotarolimus or in combination with escalating dosages of fluocinolone and evaluating the expression of MCP-1 and TF in the vessels stented with these stents. Low levels of MCP-1 or TF are considered being levels that comparable to the MCP-1 and TF levels vessels stented with stents with no drug coating. Representative experimental data is illustrated in FIG. 3A and FIG. 3B. The stented vessels can be excised 7 days post stenting and the expression of TF and MCP-1 can be evaluated via real time-PCR using porcine primers and probes, allowing the quantative measurement at transcriptional level.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A stent comprising:
(a) a predominantly cylindrical shape comprising an inner surface, an outer surface, a proximal end and a distal end;
(b) at least one polymer covering at least a portion of said inner surface, said outer surface, said proximal end, or said distal end;
(c) at least one compound capable of inhibiting smooth muscle cell proliferation dispersed within said polymer; and
(d) at least one compound capable of mitigating MCP-1 and/or TF induction dispensed within said polymer.

2. The stent according to claim 1 wherein said compound capable of mitigating MCP-1 and/or TF induction is a corticosteroid.

3. The stent according to claim 2 wherein said corticosteroid is fluocinolone.

4. The stent according to claim 1 wherein said compound capable of inhibiting smooth muscle cell proliferation is a mTOR inhibitor.

5. The stent according to claim 4 wherein said mTOR inhibitor is selected from the group consisting of sirolimus, everolimus, and zotarolimus.

6. The stent according to claim 1 wherein said stent is selected from the group consisting of vascular stents, urethral stents, biliary stents, or stents intended for use in other ducts and organ lumens.

7. The stent according to claim 1 wherein said stent has a core structure comprising a metal, a metal alloy, a polymer, a polymer blend, a polymer matrix, or combinations thereof.

8. The stent according to claim 1 wherein said polymer is selected from the group consisting of polyolefins, polyisobutylene, ethylene-alphaolefin copolymers, acrylic polymers, acrylic copolymers, ethylene-co-vinylacetate, polybutylmethacrylate, vinyl halide polymers, vinyl halide copolymers, polyvinyl ethers, polyvinylidene halides, polyacrylonitrile, polyvinyl ketones, polyvinyl aromatics, polyvinyl esters, polyvinyl amides, copolymers of vinyl monomers with each other, copolymers of vinyl monomers with olefins, acrylonitrile-styrene copolymers, polyamides, alkyd resins, polycarbonates, polyoxymethylenes, polyimides, polyethers; epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, and combinations thereof.

9. The stent according to claim 1 wherein said at least one compound capable of inhibiting smooth muscle cell proliferation is present at about 0 to 1000 μg.

10. The stent according to claim 1 wherein said at least one compound capable of mitigating MCP-1 and/or TF induction is present at about 0 to 1000 μg.

11. The stent according to claim 1 wherein said polymer and said at least one compound capable of inhibiting smooth muscle cell proliferation have a ratio of about 5:1.

12. The stent according to claim 1 wherein said polymer and said at least one compound capable of mitigating MCP-1 and/or TF induction have a ratio of about 5:1.

13. A method of forming a bioactive stent comprising the steps of:
(a) providing a stent;
(b) providing at least one polymer;
(c) providing at least one compound capable of inhibiting smooth muscle cell proliferation and at least one compound capable of mitigating MCP-1 and/or TF induction;
(d) combining said at least one compound capable of inhibiting smooth muscle cell proliferation and at least one compound capable of mitigating MCP-1 and/or TF induction with said at least one polymer to create a bioactive polymer system; and
(e) coating at least a portion of said stent with said bioactive polymer system to form a bioactive stent;

14. The method according to claim 13 wherein said stent is selected from the group consisting of vascular stents, urethral stents, biliary stents, or stents intended for use in other ducts and organ lumens.

15. The method according to claim 13 wherein said polymer is selected from the group consisting of polyolefins, polyisobutylene, ethylene-alphaolefin copolymers, acrylic polymers, acrylic copolymers, ethylene-co-vinylacetate, polybutylmethacrylate, vinyl halide polymers, vinyl halide copolymers, polyvinyl ethers, polyvinylidene halides, polyacrylonitrile, polyvinyl ketones, polyvinyl aromatics, polyvinyl esters, polyvinyl amides, copolymers of vinyl monomers with each other, copolymers of vinyl monomers with olefins, acrylonitrile-styrene copolymers, polyamides, alkyd resins, polycarbonates, polyoxymethylenes, polyimides, polyethers; epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, and combinations thereof.

16. The method according to claim 13 wherein said at least one compound capable of inhibiting smooth muscle cell proliferation is a mTOR inhibitor.

17. The method according to claim 16 wherein said mTOR inhibitor is selected from the group consisting of sirolimus, everolimus, and zotarolimus.

18. The method according to claim 13 wherein said at least one compound capable of mitigating MCP-1 and/or TF induction is a corticosteroid.

19. The method according to claim 18 wherein said corticosteroid is fluocinolone.

20. The method according to claim 13 wherein said lumen is a coronary artery.

21. The method according to claim 13 wherein said at least one compound capable of inhibiting smooth muscle cell proliferation is present at about 0 to 1000 μg.

22. The stent according to claim 13 wherein said at least one compound capable of mitigating MCP-1 and/or TF induction is present at about 0 to 1000 μg.

23. The stent according to claim 13 wherein said polymer and said at least one compound capable of inhibiting smooth muscle cell proliferation have a ratio of about 5:1.

24. The stent according to claim 13 wherein said polymer and said at least one compound capable of mitigating MCP-1 and/or TF induction have a ratio of about 5:1.
